(19) United States
(12) Patent Application Publication
(10) Pub. No.: US 2016/0030762 A1
(43) Pub. Date: Feb. 4, 2016

(54) DEVICE AND METHOD FOR TRANSCRANIAL MAGNETIC STIMULATION COIL POSITIONING WITH DATA INTEGRATION

(71) Applicant: NEUROGATE MEDICAL SYSTEMS INC., West Vancouver (CA)

(72) Inventors: Iain GLASS, West Vancouver (CA); Bruce HOSFORD, Shoreline, WA (US); Keith DOUCET, Waterloo (CA); Todd Schneider, Waterloo (CA)

(73) Assignee: Neuhorizon Medical Corporation, Vancouver, BC (CA)

(21) Appl. No.: 14/777,340

(22) PCT Filed: Mar. 17, 2014
(86) PCT No.: PCT/CA2014/050278

§ 371 Date: Sep. 15, 2015

Related U.S. Application Data
(60) Provisional application No. 61/790,003, filed on Mar. 15, 2013.

(51) Int. Cl. A61N 2/02

(52) U.S. Cl. CPC A61N 2/02

(57) ABSTRACT

There is disclosed device and method provide efficient, comfortable and accurate positioning of a treatment device relative to a specific cranial anatomical location of a subject. In an embodiment, the method comprises: placing a fixed-position locating device on the cranium of the subject; placing an adjustable positioning cap on the cranium of the subject; loading a subject-specific calibration for treatment of a specified cranial anatomical location; determining a three-dimensional position of the fixed-position locator device relative to the adjustable positioning cap; and calibrating the location of the positioning cap by adjusting the positioning cap until it is aligned with the specified cranial anatomical location.

### TECHNICAL FIELD

The present disclosure relates to accurate positioning of a treatment device relative to a subject who is undergoing treatment with the device.

### BACKGROUND

Transcranial magnetic stimulation (TMS) is a noninvasive method used to cause depolarization or hyperpolarization in the neurons of the brain. TMS uses electromagnetic induction generated by an induction coil to induce weak electric currents using a rapidly changing magnetic field. This can cause activity in specific or general parts of the brain with minimal discomfort, allowing the functioning and interconnections of the brain to be studied, or for the purposes of treatment of some brain disorders.

TMS therapy is currently Federal Drug Administration (FDA) approved for treatment of some forms of drug resistant depression. It is also being studied as a possible treatment for a wide range of other central nervous system disorders including epilepsy, schizophrenia, Parkinson's Disease, Tourette's Syndrome, Amyotrophic Lateral Sclerosis, Multiple Sclerosis, Alzheimer's Disease, Attention Deficit/Hyperactivity disorder, obesity, bipolar disorder, post-traumatic stress disorder (PTSD), anxiety disorders, obsessive-compulsive disorder (OCD), pain, chronic pain, stroke rehab, tinnitus, addiction and withdraw disorders, insomnia, traumatic brain injury, seizure therapy and other central nervous system (CNS) disorders that may be treated by the application of a magnetic field to specific regions of the brain. Each of these disorders has a specific anatomical treatment location or locations that may be targeted with the TMS pulses. US patent application 2005/0148808 provides an extensive list of disorders and typical treatment locations.

An important aspect of TMS treatment is the repeatable, accurate placement of the induction coil at the desired treatment location. A recent study by C. Nauczyciel et al. (Assessment of standard coil positioning in transcranial magnetic stimulation in depression, Psychiatry Research 186 (2011) 232-238) highlighted the importance of proper coil placement and concluded that accurate positioning of the coil is mandatory to conduct reproducible and reliable studies.

Traditional approaches to TMS treatment have used placement methods that do not always result in accurate placement of the magnetic pulses relative to the desired anatomical treatment locations on the subject. Newer approaches often use complex and expensive equipment (e.g., robotics) that may not be practical or cost-effective.

A widely used approach for placement of the TMS coil is a manual method where the location on the skull which activates the subject's motor threshold (the motor threshold location or MTL) is found through trial and error. Once this location is found, it is marked with ink and the coil is moved (e.g. 5 cm in the anterior direction) to find the TMS therapy point (TTP), which is also marked with ink so it can be found again for future reference. This approach, along with other variations is problematic because it is inaccurate; does not reference to the underlying brain locations; and the ink marks made are not permanent. Thus, the entire procedure may need to be repeated for each therapy session. Furthermore, in some current treatment protocols, the TMS coil is held manually in-place. This is impractical because TMS coils can be heavy and as a result operator fatigue and/or incorrect planar placement relative to the subject's skull may result in less effective TMS therapy than could otherwise be realized.

US patent publication 2005/0148808 and U.S. Pat. No. 7,651,459 describe a system where the subject's head is held in a known, fixed position by a headset assembly and the TMS coil is held fixed using a mast and gantry at the TTP relative to the subject's head. US patent publication 2006/0122496 A1 also discloses a system that holds the subject's head in a fixed position. These systems address operator fatigue and improve placement accuracy, but restraining the subject's head can result in discomfort for the subject potentially decreasing subject compliance.

US patent publication US 2009/0227830 describes an improvement on US 2005/0148808 which allows the subject's head to be flexibly positioned. However, this system still requires the subject's head to be held in place using padded inserts during TMS therapy.

An alternative to holding the subject's head stationary is to use a stereotactic vision system to track subject head movement. Commercially available systems from ANT Neuro and Northern Digital Inc. use this technique. A similar approach is disclosed in U.S. Pat. No. 7,854,232 B2. These systems use an infrared camera to locate the position of reflective spheres that are affixed to the subject and the TMS coil. With some systems (e.g., SmartMove) the operator can receive real-time visual feedback regarding coil placement relative to the TTP and make necessary corrections. These systems provide accurate positioning and improved subject comfort, but they do not always address operator fatigue. They are also very costly. Additionally, SmartMove and U.S. Pat. No. 7,087,008 B2 address operator fatigue by using a robotic arm to hold and manipulate the TMS coil. However, this further increases cost and may lead to a subject environment that is overly complex and could be viewed as threatening by some subjects.

Another system referred to as the “BrainVoyager system” utilizes ultrasonic emitters mounted on the subject and the TMS coil at known reference positions. Using three microphones built into a positioning system and time of flight measurements on the ultrasonic signals, the relative location of the subject, the coil and the TTP may be found and tracked in real-time. This approach may be cumbersome in a clinical environment where attaching the transducers to the subject will take time and may be uncomfortable for the subject. It also does not address operator fatigue.

Patent application US2010/0249577 A1 discloses a positioning method that uses the magnetic field generated by the coil to implement a tracking system. However, this approach requires that the magnetic field from the coil be calibrated.

In addition to accurate positioning, recent advances in combining TMS with electroencephalogram (EEG) measurements have shown that EEG measurements can be very useful for advanced detection of potential seizure and on-going monitoring of treatment progress (for example, see US patent application 2011/0119212 A1). Current EEG systems for use with TMS are complex, requiring a significant setup that is not integrated with the TMS setup and positioning. This increases the time required for treatment setup which prohibits the use of EEG data collection and analysis in high volume clinical applications.

Thus, in summary, conventional, manual approaches to TMS coil placement are inaccurate and do not address operator fatigue. As well, other positioning systems proposed to date are both complex and expensive; or require the subject's head to be held stationary (and therefore negatively impact subject comfort and acceptance of treatment.

What is needed is a solution that addresses at least some of the limitations outlined above.

### SUMMARY

The present disclosure relates to a system and method for accurate positioning of a treatment device relative to a subject who is undergoing treatment with the device. More specifically, the disclosure relates to a system and method for accurate and repeatable placement of one or more transcranial magnetic stimulation (TMS) coils relative to specific anatomical locations of the subject, including the cranial, central nervous system, spinal cord, and peripheral nervous system, while ensuring that the subject is comfortable during treatment and that the location procedure is efficient and robust with respect to subject movement and overall subject positioning.

In an embodiment, the system and method may also be used for accurate placement of electroencephalograph (EEG) electrodes or alternatively transcranial direct current stimulation (tDCS) devices for treatment of specific anatomical locations.

The present positioning system and method is adapted to ensure that the treatment device is positioned accurately over the treatment location, and does not move during TMS treatment such that the treatment location can be targeted for the desired length of time, at the desired level of power. Additionally, the present positioning system and method is well suited to high volume clinical TMS treatments as the positioning setup procedure is low cost, simple and efficient to operate, as well as accurate and repeatable for a given subject. To ensure subject comfort and compliance with the TMS therapy protocols, the present positioning system and method is comfortable for the subject, allowing some freedom of movement. Finally, the present positioning system and method is adaptable to work with all sizes and shapes of subjects, and not be impeded by hair or other obstacles.

In an aspect, the system and method disclosed and claimed herein comprises a positioning cap that can be provided with or without integrated EEG electrodes; a locator device, a positioning device; and a location sensor which is used to locate the positioning device and locator device in three-dimensional space.

In various other aspects, the present system and method includes the following features: (1) a positioning cap used to reference the TTP for TMS therapy which incorporates alignment guides (for example locator pins) that mate with corresponding alignment receptacles (for example locator holes) in the TMS coil and the positioning device. The alignment guides are designed to keep the coil and the positioning device in the desired location relative to the subject's skull;

(2) A positioning device that can be placed on the positioning cap in a known and repeatable location via alignment guides in the positioning cap and locator holes in the bottom of the positioning device;

(3) A locator device which is used to locate the subject in three-dimensional space relative to a location detector. This device can be eyeglasses, a headband or similar;

(4) A positioning cap that is worn by the subject during treatment. The cap incorporates alignment guides (for example locator pins) which are designed to keep the coil in the correct location and help to reduce operator fatigue;

(5) A positioning cap that optionally integrates an array of EEG electrodes arranged at known and fixed locations relative to the TTP and external electrodes. It is also possible to integrate the positioning device electronics and/or the EEG front-end electronics directly into the positioning cap;

(6) A TMS coil with alignment guides (for example locator holes) that mate with corresponding locator devices (for example pins) on the positioning cap to repeatedly position the TMS coil into a desired location relative to the TTP. In another embodiment, the coil may also interface with an arm that will hold the coil in the desired location using the location pins in the cap. This ensures precise and repeatable measurements, significantly improving data collection integrity.

(7) Selectable processing of the EEG electrodes based on which electrodes provide valid EEG signals and the use of the these EEG electrode signals to assess the proximity of the TMS positioning cap to the subject's skull;

(8) Storage of relative location of positioning cap/positioning device and locator device in a control unit for later recall; and

(9) The possibility of making the positioning cap, positioning device and/or the EEG data transmission passive or wireless

### BRIEF DESCRIPTION OF THE DRAWINGS

FIG. 1 illustrates a TMS treatment system in accordance with an embodiment.

FIG. 2 illustrates a block diagram of a TMS treatment system in accordance with an embodiment.

FIGS. 3A and 3B show an exemplar locator device, locator glasses, in accordance with various embodiments.

FIG. 4 shows another exemplar locator device, a headband, in accordance with an embodiment.

FIGS. 5A and 5B show an illustrative positioning cap without integrated EEG electrodes in accordance with an embodiment.

FIGS. 6A and 6B show another illustrative positioning cap with integrated EEG electrodes in accordance with an embodiment.

FIG. 7 shows a bottom view of the positioning cap of FIG. 6 with integrated EEG electrodes.

FIGS. 8A and 8B show alternative views of an illustrative positioning device in accordance with an embodiment.

FIGS. 9A and 9B show an illustrative TMS coil in accordance with an embodiment.

FIG. 10 shows illustrative flow charts for calibration, treatment setup and treatment phases of a TMS therapy session using the device and method disclosed herein.

FIGS. 11A and 11B show an illustrative positioning guidance screen in accordance with an embodiment.

FIG. 12 shows flow charts for calibration, treatment setup and treatment which incorporate EEG electrode data integration using the device and method disclosed herein.

FIG. 13 shows an illustrative EEG electrode calibration and setup screen in accordance with an embodiment.

FIG. 14 shows flow charts for positioning system operation during the calibration, treatment setup and treatment phases of a TMS therapy session using the device and method disclosed herein.

Like reference numerals indicate like parts throughout the diagrams.

### DETAILED DESCRIPTION

As noted above, the present disclosure relates to accurate positioning of a treatment device relative to a subject who is undergoing treatment with the device.

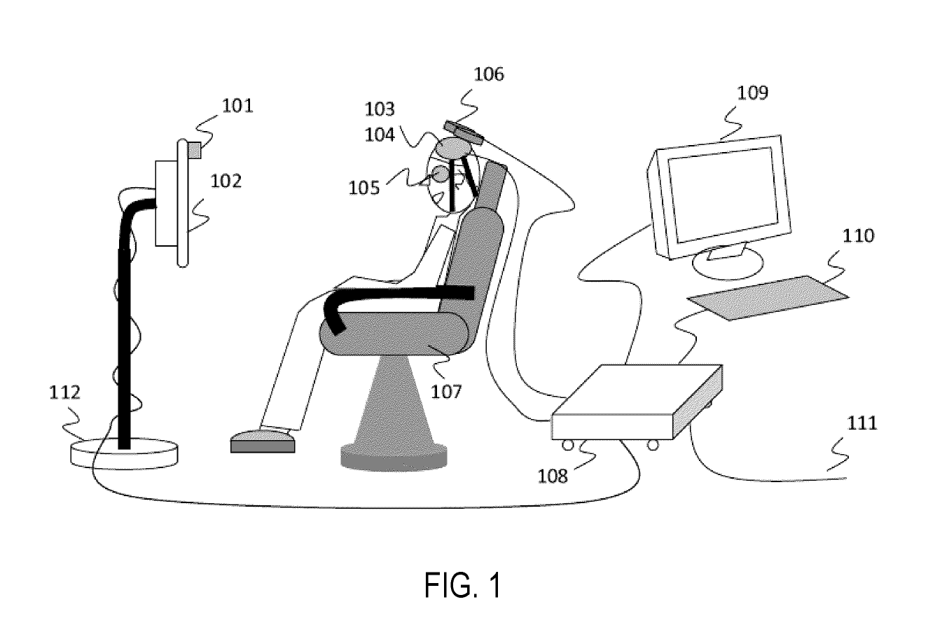

FIG. 1 illustrates a TMS Treatment System in accordance with an embodiment. In this illustrative example, the TMS Treatment System incorporates positioning cap (103) which may have integrated EEG electrodes; a positioning device (104) placed on the positioning cap (103); locator device (105); location sensor (101); TMS coil (106); subject monitor (102) and stand (112); treatment chair (107); control unit (108); control monitor (109) and keyboard (110); and a network connection (111). The operation of the system will be described in detail below.

During a therapy session, the subject is seated in the treatment chair (107). The subject views the subject monitor (102) that is mounted on a stand (112). The treatment monitor is used to relay information to the subject about the progress and status of the therapy session. It can also be used for entertainment purposes during the therapy session. A location sensor (101) may be advantageously mounted on the same stand as the subject monitor (as shown) or, it may be mounted on a separate stand. If the chair is in a reclining position, the subject monitor (102) and the location sensor (101) may be advantageously mounted on a stand that allows repositioning so the subject can view the monitor and the location sensor has an unobstructed view of the subject. In an alternative implementation, the stand may be mounted to the treatment chair (107).

As detailed below, there are three phases to the TMS therapy session: calibration, treatment setup and treatment. The calibration phase is done only when the TMS therapy point (TTP) needs to be located or re-located. The treatment setup and treatment phases occur each time the subject receives TMS therapy. The subject wears a positioning cap (103) for all phases of the therapy session. The positioning cap allows the TMS coil (106) to be accurately positioned relative to the TMS therapy point (TTP) for the treatment portion of the TMS therapy session. It may also incorporate EEG electrodes as described below. During the calibration and treatment setup phases, the subject wears a locator device (105), as described further below, and a positioning device (104) is placed on the positioning cap (103).

A control unit (108) provides overall system control as well as the signals necessary to drive the TMS coil to provide the desired therapy. Control unit (108) optionally receives EEG signals from the positioning cap (103) and processes the EEG signal as outlined in detail below. A control monitor (109) which may incorporate a touch screen and an optional keyboard (110) allows the operator (e.g. a TMS technician or TMS/EEG technician) to configure the TMS Therapy System and provide the desired therapy to the subject. A wired or wireless network connection (111) allows the control unit (108) to communicate with other devices (e.g., an electronic medical records system that provides subject information, including per subject therapy information).

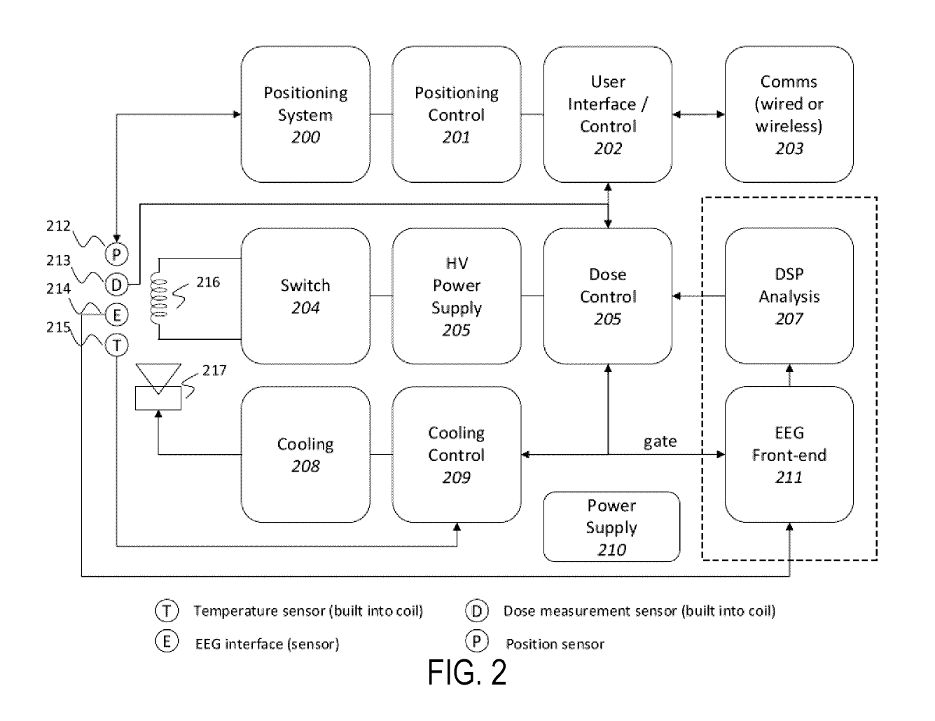

FIG. 2 shows a block diagram of the TMS Treatment System. In an embodiment, the TMS Treatment System comprises a positioning system (200) which incorporates positioning control (202); a user interface (202); a positioning sensor (or sensors—212); and a communications interface (203). For the application of TMS therapy, it incorporates dose control (205); a high voltage power supply (206); one or more high voltage switches (204); one or more TMS coils (216); and a means for measuring the TMS dose (213). For coil and system cooling, the TMS system incorporates cooling control (209); a cooling system (208); a means (217) for cooling the coil; and a temperature sensor (215). For monitoring and safety, the TMS system may optionally incorporate an EEG subsystem that is comprised of EEG sensors (214); a multichannel EEG front-end (211); and a digital signal processing (DSP) analysis block (207) which is used for EEG signal processing and dose calculation. Power for the entire TMS system is provided by the power supply subsystem (210).

In an illustrative embodiment, the TMS Treatment System includes various subsystems: (1) TMS control, (2) cooling, (3) positioning, (4) user interface, (5) EEG monitoring, (6) communications and (7) power supply.

The TMS control subsystem is comprised of a dose control block (205) that controls the intensity and timing (duration, repetition rate and duty cycle) of the magnetic pulses that are delivered by the TMS coil (216; also 106 in FIG. 1). A high voltage power supply (206) provides the current and voltage necessary to drive the TMS coil via a switch (204) that is controlled via the dose control block (205). The user interface (202) allows the TMS technician providing the therapy to adjust the key parameters of the TMS therapy session or load a predetermined therapy plan that may be provided over a network connection via the communications interface (203).

The cooling subsystem provides cooling to the TMS coil to ensure that the coil does not overheat. The cooling subsystem includes cooling control (209), and a cooling means (209 and 217). A temperature sensor (215) provides temperature feedback to the cooling control block.

The positioning subsystem is comprised of positioning control (201) and the positioning system (200), along with position sensing (212). These elements implement the control and system portions related to the positioning cap (104), positioning device (104) and the locator device (105) outlined above. Details on their operation are described below.

The user interface (202) provides a means for the TMS technician to control the operation of the TMS Treatment system. It implements the software and hardware control that is required for the TMS technician to interact with the system using the control monitor (109) and the keyboard (110). A communications interface (111) provides a means for the user interface to send and receive data and/or control information remotely.

The EEG monitoring subsystem receives data from the EEG electrodes that are integral to the positioning cap (see below for details). This data is provided to a multi-channel EEG front-end (211) and the output of the front-end is processed by the DSP analysis block (207). The multi-channel EEG (211) front-end is resistant to the artifacts introduced by the high-magnetic fields experienced during TMS therapy sessions as described in U.S. Pat. No. 6,571,132 B2 and J. R. Ives et al., Electroencephalographic recording during transcranial magnetic stimulation in humans and animals, Clinical Neurophysiology 117 (2006) 1870-1875 and The Oxford Handbook of Transcranial Stimulation, Oxford Handbooks, by Wassermann, Epstein and Ziemann (Editors) pages 595-596. The DSP analysis block (207) analyzes the EEG data to determine if the subject is experiencing epileptiform discharges or any similar condition which could indicate that seizure is likely (e.g., kindling). If seizure is likely, therapy may be stopped or prevented from starting. Additionally, the EEG data is monitored to assess the treatment progress; determine if the TMS therapy is resulting in the desired changes in the EEG waveforms; and to determine the proximity of the positioning cap to the subject's skull. See US patent application 2011/0119212 A1 for possible techniques that could be used for EEG monitoring of treatment progress.

The power supply (210) converts input AC line voltage to the voltages and currents as required by all of the other subsystems that comprise the TMS Treatment System.

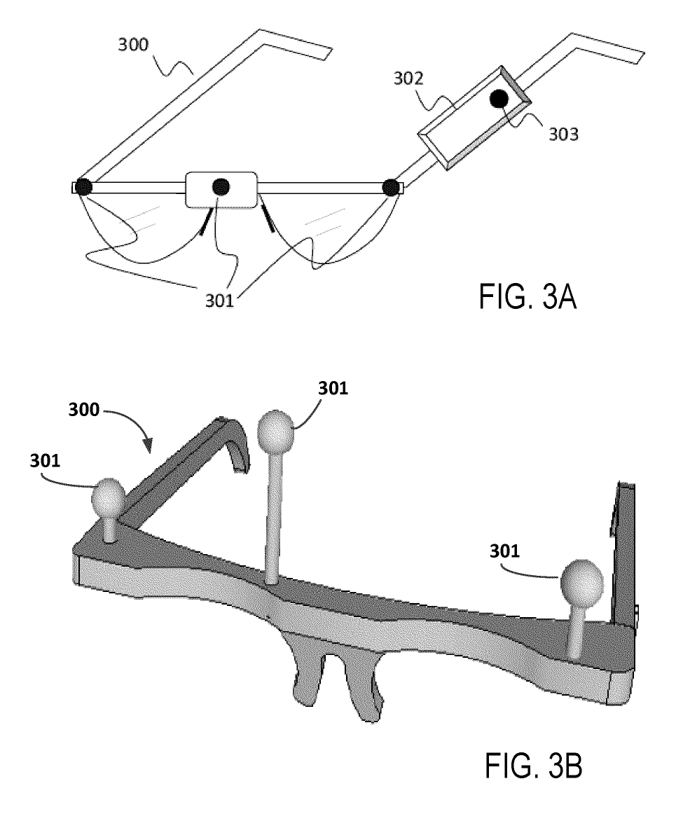

FIGS. 3A and 3B show an exemplar locator device (105), for example locator glasses. In an embodiment, the locator glasses includes a frame (300); three or more passive optical targets or active emitters (301); an electronics subsystem for driving the emitters (302); and an optional connector for powering and communicating with the emitters (303). In the case of active emitters, if the connector (303) is not provided, the glasses will have an internal power source (e.g., a battery) and will operate wirelessly, as illustrated in FIG. 3B.

As described below, the locator device is used to locate the subject in three-dimensional space. The glasses consist of a lightweight frame (300) that fits comfortably on the subject in a repeatable location during the calibration and treatment setup phases (as outlined below). Different sizes of frames may be provided to ensure a snug fit and therefore provide a repeatably measurable location of the subject's head. The glasses have at least three passive optical targets that are tracked by an external imaging system, or emitters (301) that are controlled via control electronics (302). A connector (303) allows for wired control of the emitters. The locator device may also be provided with a wireless link. In this case, the control electronics (302) will have an integral power source (e.g., a battery) and the connector (303) is not required, although it may still be present for recharging the battery and communicating with the control electronics for the purposes of re-configuration. The operation of the control electronics and emitters is described below.

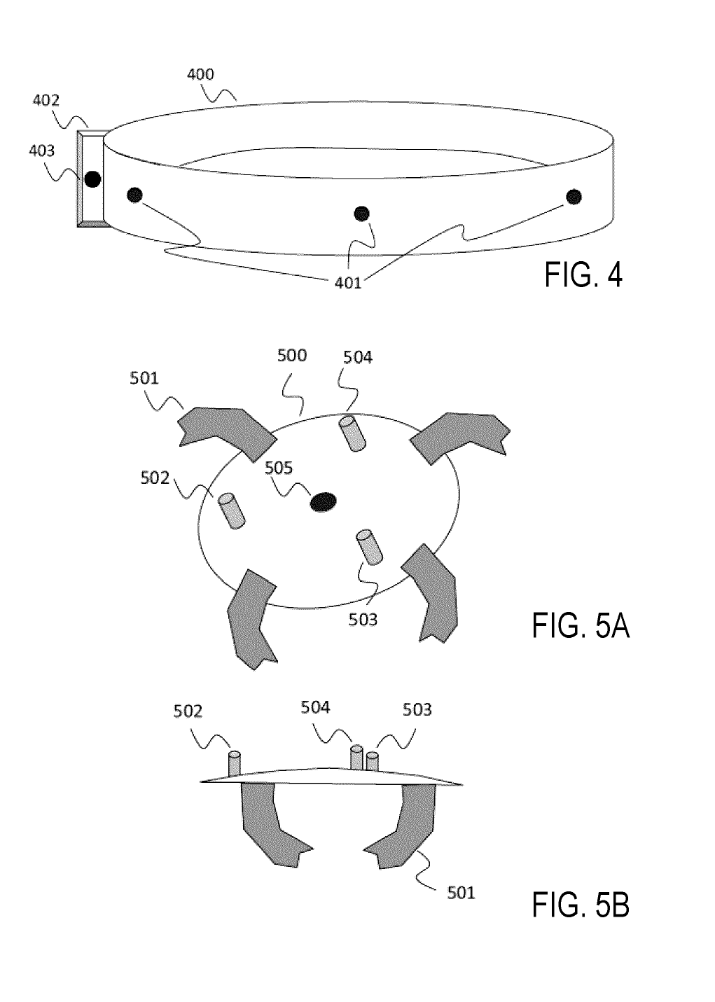

A number of variations of the locator device are possible. For example, FIG. 4 shows another exemplar locator device, a headband. In an embodiment, the headband includes a headstrap (400); three or more passive optical targets or active emitters (401); an electronics subsystem for driving the emitters (402); and an optional connector for powering and communicating with the emitters (403). If the connector (403) is not provided, the headband will have an internal power source (e.g., a battery) and will operate wirelessly. For repeatable positioning, the headband can rest on the subject's ears, for vertical alignment, and rotated until the middle emitter lines up with the center of the subject's nose, for horizontal alignment.

Similar to the glasses, there are passive optical targets or active emitters (401), control electronics (402) and a connector (403). The headstrap (400) takes the place of the frame (300). The essential elements of any locator device are (1) at least three optical targets or emitters; (2) control electronics to control independently the emitters over a wired or wireless connection (in the case of active emitters); and (3) a physical form that allows the locator device to be comfortably worn by the subject in a repeatable position. Different sized locator devices may be provided as required to ensure that the position of the locator device is both accurate and precise for each subject.

FIGS. 5A and 5B show an illustrative positioning cap without integral EEG electrodes. In an embodiment, the positioning cap includes a cap base (500), one or more mounting straps (501); alignment devices (for example pins) (502, 503, 504); and a target location (505). This type of positioning cap would be used for TMS therapy sessions where the doctor or clinician prescribing treatment determined that it was not necessary to monitor EEG waveforms during treatment. During the treatment session, the positioning cap is worn by the subject with the cap base (500) against their head; the target location (505) over the TMS therapy point (TTP); and the mounting straps (501) firmly affixing the positioning cap to the subject's head so it will not move during the TMS therapy session. The positioning cap has at least two alignment devices (for example locator pins) (502, 503, 504) that mate with corresponding receptacles (for example locator holes) on the positioning device and also on the TMS coil. The purpose of the locator pins and holes is to fix the location of the positioning cap relative to the positioning device and the TMS coil. The locator pins and holes also provide assistance for the positioning of the TMS coil, thereby reducing operator fatigue.

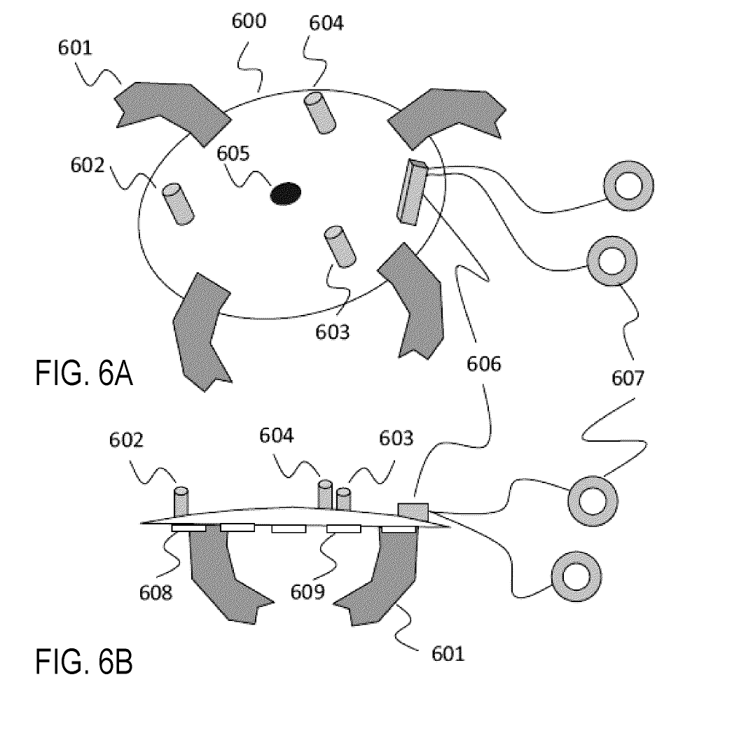

FIGS. 6A and 6B show another illustrative positioning cap (600) with integrated EEG electrodes. In an embodiment, the positioning cap includes the cap base (600), at least two mounting straps (601); at least two alignment devices (for example locator pins) (602, 603, 604); a target location (605); an EEG connector (606) that can connect to optional external EEG electrodes (607); and an array of EEG electrodes (608, 609) on the bottom of the cap base. Similar to the positioning cap without EEG electrodes, this positioning cap has mounting straps (601), a target location (605) and locator devices (for example pins) (602, 603, 604). The cap also has a connector (606) that receives signals from the EEG electrodes (608, 609) integrated into the positioning cap (600). The connector (606) also connects to optional external EEG electrodes (607) that can be placed elsewhere on the subject. The connector (606) delivers the signals from the integrated array of EEG electrodes (608, 609) and the external EEG electrodes (607) to the EEG front-end (211) located in the control unit (108). Different sized positioning caps may be provided as required to ensure that the fit of the positioning cap is both accurate and precise for each subject.

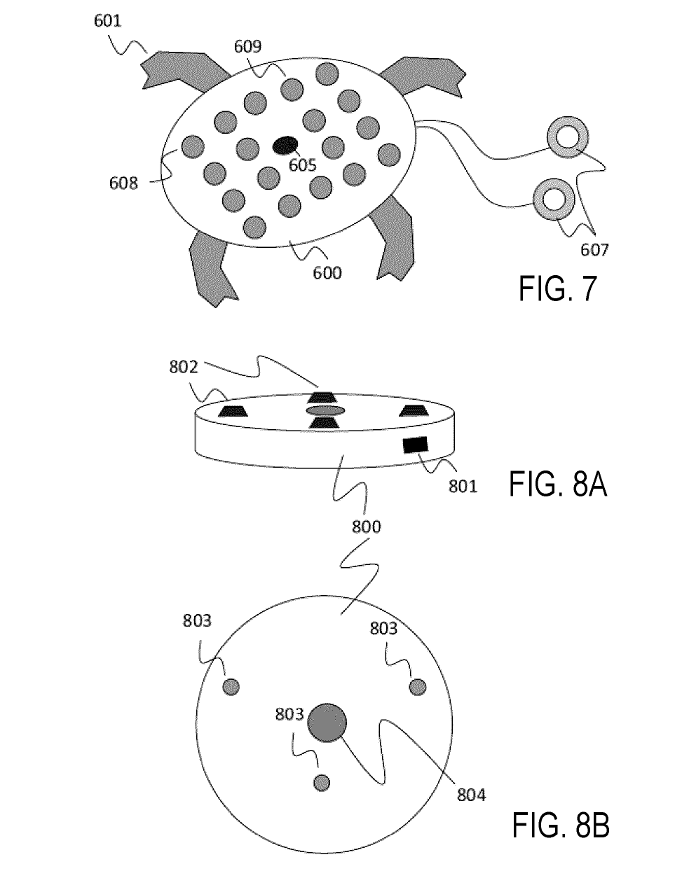

FIG. 7 illustrates the bottom of the TMS positioning cap (600) which goes against the subject's head. In an embodiment, the positioning cap includes a cap base (600), at least two mounting straps (601; a TTP target (605); an EEG connector (606—see FIGS. 6A and 6B) that connects to optional external EEG electrodes (607); and an array of EEG electrodes (608, 609) on the bottom of the cap base. Shown is the array of EEG electrodes (608, 609); the mounting straps (601); the external EEG electrodes (607); and the target location (605).

The EEG electrodes (608, 609) are configured in an array and may be integrated into the cap or snap into pre-determined locations on the cap. The EEG electrodes will be compatible with TMS therapy protocols in that they will withstand the high magnetic fields generated by the TMS therapy without causing subject discomfort or injury. As described in U.S. Pat. No. 6,571,132 B2 and J. R. Ives et al., Electroencephalographic recording during transcranial magnetic stimulation in humans and animals, Clinical Neurophysiology 117 (2006) 1870-1875, a conductive plastic electrode with silver-silver chloride (Ag—Ag/Cl) applied provides excellent recording characteristics and is compatible with TMS therapy. Other approaches for making TMS compatible EEG electrodes are well-known in the art. For example, see The Oxford Handbook of Transcranial Stimulation, Oxford Handbooks, by Wassermann, Epstein and Ziemann (Editors) page 595. Either wet or dry EEG electrodes, as best suited to the treatment needs may be used. It will be readily recognized by those skilled in the art that the number of EEG electrodes can be varied as required for specific measurement purposes.

All or a portion of the EEG front-end electronics (211) may be integrated into the positioning cap provided the electronics are designed and housed in a manner that is compatible with the high magnetic fields experienced during TMS therapy. It is also possible to replace the wired transmission of the EEG signals from the array of EEG electrodes (608, 609) and the external EEG electrodes (607) with a wireless link to the control unit (108). In this case, the positioning cap would incorporate an electronics subsystem with an integrated power source (e.g., a battery) and the connector (606) is not required, although it may still be present for recharging the battery and communicating with the integrated electronics subsystem for the purposes of re-configuration.

FIGS. 8A and 8B show an illustrative positioning device (800). In an embodiment, the positioning device includes a case (800); at least three optical targets or emitters (802) and a connector (801) for powering and controlling the emitters; and at least two locator receptacles (for example holes) (803). If the connector (803) is not provided, the positioning device will have an internal power source (e.g., a battery) and will operate wirelessly. As described below, the positioning device is used to locate the TTP relative to the locator device in three-dimensional space. The positioning device has at least three optical targets or emitters (802); an electronics subsystem integral to the positioning device for independently driving the emitters (in the case of active emitters) (802); and a connector (801) for powering and communicating with the emitters (802). The bottom side of the positioning device has locator holes (803) that mate with the locator pins on the positioning cap (502, 503, 504 or 602, 603, 604). A through-hole (804) is also provided so the target location (505 or 605) is visible during the calibration and treatment setup phases (as outlined below).

Wired communication with the positioning device can be replaced with wireless communication. In this case, the positioning device would contain an integral power source (e.g., a battery) and the connector (801) is not required, although it may still be present for recharging the battery and communicating with the integrated electronics subsystem for the purposes of re-configuration. Additionally, the entire positioning device could be advantageously integrated into either of the positioning caps shown in FIGS. 5A and 5B, or FIGS. 6A and 6B.

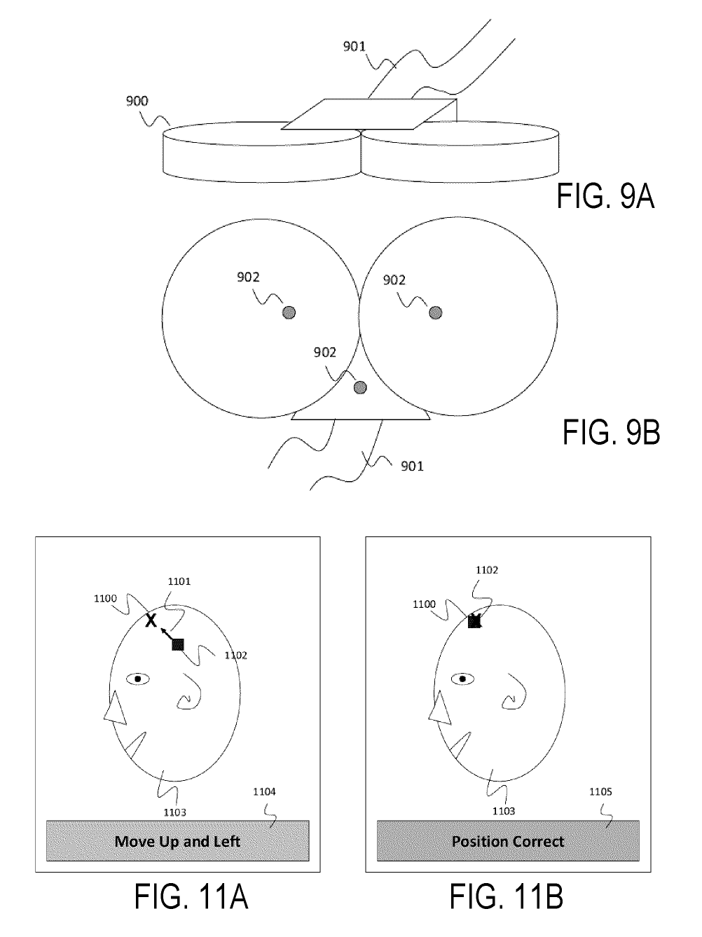

FIGS. 9A and 9B show an illustrative TMS coil (900), also labeled 106 in FIG. 1, that will be used with the TMS system. In an embodiment, the TMS coil includes a case (900); a cable (901) to power the windings inside the case and provide control signals to/from the sensors inside the case; at least two locator receptacles (for example holes) (902).

In an embodiment, a cable (901) provides power to the coil windings located inside the coil casing as well as providing control and communication with the sensors (e.g. temperature) and user controls (e.g., a switch or indicator light) that may be integrated into the TMS coil. The bottom face of the TMS coil (that will face the subject's head during a TMS therapy session) contains as least two locator receptacles or holes (902). These locator holes mate with the alignment devices (for example pins) on the positioning cap (502, 503, 504 or 602, 603, 604) to ensure proper positioning of the coil relative to the TTP. Although FIGS. 9A an 9B show a “FIG. 8” TMS coil, it will be readily recognized to those skilled in the art that other TMS coil types (e.g., circular, angled FIG. 8 or coil arrays used for deep brain TMS) can be used in a similar arrangement.

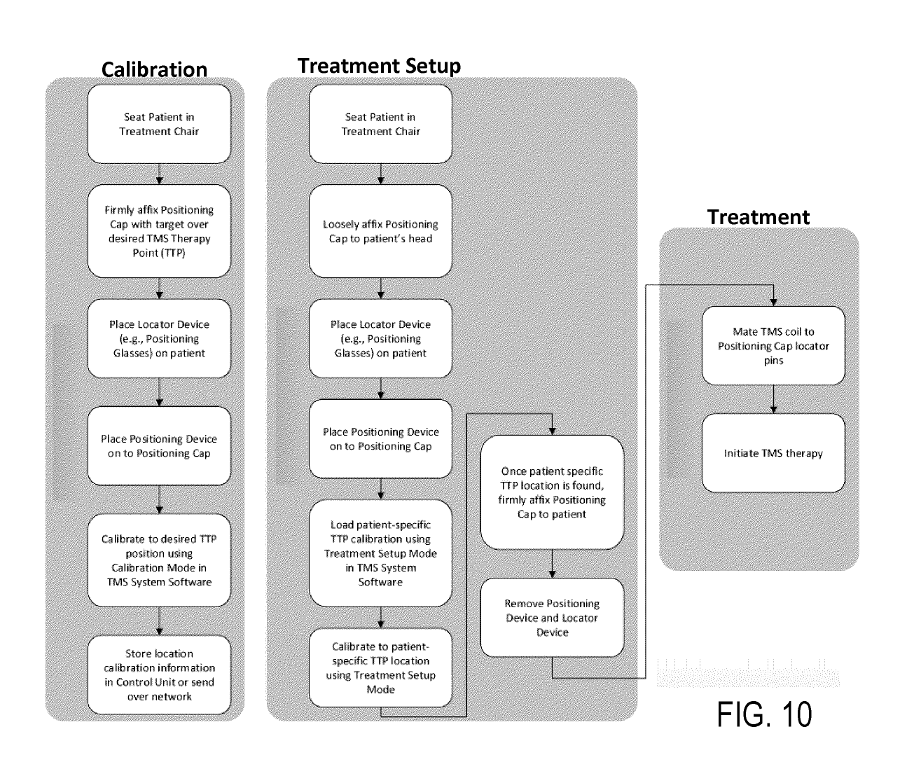

FIG. 10 describes how the positioning system with EEG data integration is used in a TMS therapy session. There are three distinct phases: calibration, treatment setup and treatment.

The calibration phase is only done when it is necessary to calibrate or re-calibrate the TMS therapy point (TTP) for a specific subject. At the initiation of the calibration phase, the TTP for the subject to be calibrated is known and is either marked on the subject's scalp using ink or located via some other means. As shown in FIG. 10, the first step is to seat the subject in the treatment chair (107); then the positioning cap (103 also 500 or 600) is firmly affixed to the subject with the target location (505 or 605) located directly over the known TTP. Next, the locator device (105, FIG. 3 or FIG. 4) is put onto the subject and the positioning device (800) is placed on the positioning cap (103 also 500 or 600). As noted above, the positioning device may be integrated into the positioning cap, in this case only the positioning cap needs to be placed on the subject. Then, using the location sensor (101), control monitor (109), control unit (108) and optionally the keyboard (110), the three-dimensional position of the locator device and the positioning device are computed and stored in the control unit (108). Using the position of the locator device, the position of the positioning device (and hence the TTP) relative to the locator device is computed and stored in the control unit (108). If desired all of the resulting positioning information may then be sent across the network using the network connection (111) for storage as part of the subject's electronic medical record and/or treatment records.

The treatment setup phase is used before every TMS therapy session. At the initiation of the treatment setup phase, the patent is seated in the treatment chair (107). Then, the positioning cap (103 also 500 or 600) is loosely affixed to the subject at a location that is estimated by the TMS technician to be close to the TTP. Then, the locator device (105, FIG. 3 or FIG. 4) is put onto the subject and the positioning device (800) is placed on the positioning cap (103 also 500 or 600) such that the alignment devices (for example pins) (502, 503, 504 and 602, 603, 604) mate with the corresponding receptacles (for example locator holes) (803).

Next the subject specific TTP calibration information is loaded by the control unit (108). The control unit may optionally retrieve this information from the subject's electronic medical record or treatment record using the network connection (111). Using the location sensor (101) and techniques that are outlined below, the position of the locator device (105, FIG. 3 or FIG. 4) in three-dimensional space is calculated.

Next, the position of the positioning device in three-dimensional space is calculated and compared to the calibrated positioning information that was loaded by the control unit. The TMS technician is shown a graphical display (FIG. 11) that illustrates the change in position required to bring the positioning device (and therefore the positioning cap) into a position where the target location (505 or 605) is directly over the subject specific TTP location. Once the TMS technician has moved the target location to the calibrated TTP location, the graphical display indicates this state.

Then, the TMS technician can firmly affix the positioning cap to the subject using the mounting straps and remove the positioning device from the positioning cap. As noted previously, the positioning device may be integrated into the positioning cap. In this case, the positioning device does not need to be removed from the subject. Once the positioning cap is firmly affixed to the subject with the target location over the subject specific TTP; the locator device is removed; if required, the positioning device is removed; and the treatment setup phase is complete.

In the treatment phase, the TMS coil (106 and 900) is positioned so that the location receptacles (for example holes) (902) mate with the alignment devices (for example pins) (502, 503, 504 and 602, 603, 604). Then, TMS therapy is initiated using the control unit (108), control monitor (109) and optionally the keyboard (110).

FIGS. 11A and 11B show an example of the graphical display that is used to guide the TMS technician in placement of the target location (505 or 605) over the subject-specific TTP. In an embodiment, the graphical display includes a schematic representation of the subject (1103) with a TMP therapy point or TTP (1100) shown using a symbol (in this case an “X”) and the current target location is shown using a different symbol (1102). An indication of the movement required to align the TTP with the target location for the specific subject is provided (1101), along with written instructions regarding the movement (1104). An example of written instructions would be “Move up and left”. When the subject specific TTP (1100) and the target location (1102) are aligned the direction indication will disappear and the written instructions (1105) will change text (e.g. “Position correct”) and color to indicate that alignment has been achieved.

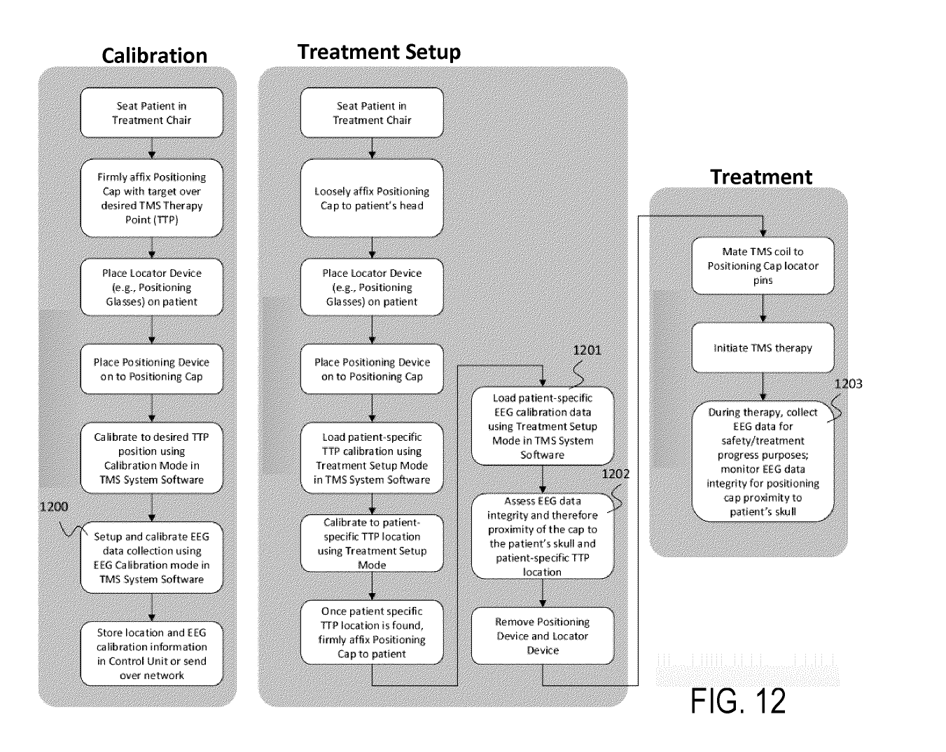

If EEG electrodes are integrated into or used in combination with the positioning cap (see FIGS. 6A and 6B, and FIG. 7), a step for the setup and assessment of EEG sensor data integrity will be incorporated into the calibration, treatment setup and treatment phases of the TMS therapy session as shown in FIG. 12.

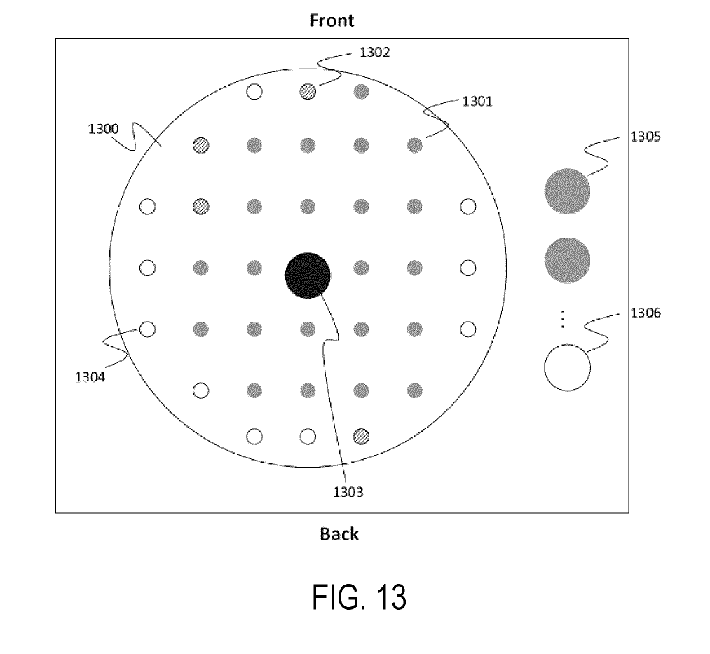

In the calibration phase, the added step (1200) includes setup and calibration of the EEG electrodes in the positioning cap. Once the TMS/EEG technician has placed the positioning cap and firmly affixed it to the subject with the mounting straps, the TMS/EEG technician will setup and calibrate the EEG electrodes. Once this setup is complete, data from the EEG electrodes will be processed by the control unit (108) and assessed to determine if they represent valid data (i.e., it contains valid EEG waveforms). This processing will be carried out by the DSP analysis block (207). If necessary, the technician will examine any suspect waveforms and the associated EEG waveforms that are not providing valid EEG signals. A screen like the one shown in FIG. 13, which is described below, will be used guide the technician. Because of anatomical and other differences between subjects, not all EEG sensors in the array on the positioning cap may provide valid signals. However, if a sufficient number of EEG sensors, covering a sufficient area under the positioning cap are providing valid data, the setup of the EEG electrodes will be deemed complete and the setup data (EEG sensors providing valid data, EEG sensors providing suspect data and EEG sensors providing invalid data) will be logged to the control unit and saved as part of the subject-specific calibration information. Additionally, representative EEG waveforms for the sensors providing valid and suspect data may be saved as part of the subject-specific EEG calibration information. All of the calibration information may be sent over the network connection (111) and stored remotely as part of a subject electronic medical record or treatment history.

In the treatment setup phase, the added steps (1201 and 1202) load the subject-specific EEG calibration data into the control unit (108). This data is then compared with the current EEG waveforms to ensure that similar results for the EEG electrode signal integrity are obtained across different TMS therapy sessions for a specific subject. The control unit (108) in combination with the DSP analysis block (207) completes calculations that compare the EEG electrode results for the current treatment session with the subject-specific EEG calibration data that has been loaded into the control unit (108). As noted above, the subject-specific EEG calibration data may be loaded over the network connection (111). At the conclusion of the treatment session, the subject-specific EEG calibration data may be updated, under control of the TMS/EEG technician, to include any changes in EEG electrode signal integrity or validity that have occurred during the treatment session. During the treatment setup phase, the TMS/EEG technician may also make adjustments to the EEG electrodes using a setup screen like the one shown in FIG. 13 in an effort to obtain consistent EEG electrode setup results across different treatment sessions.

In the treatment phase, the additional step (1203) collects EEG data for safety monitoring and treatment progress purposes. EEG electrode data is also monitored to assess the proximity of the positioning cap to the subject's skull. This is accomplished by processing data from the EEG electrodes through the EEG front-end (211) and then applying computations in the DSP analysis block (207) as required. EEG electrode data collected through-out the session is compared to EEG calibration data that was loaded during the treatment setup. Again, these comparisons are implemented in the DSP analysis block (207). Additionally, EEG waveform data at the beginning of the treatment session is stored as baseline data in the control unit (108). Through-out the treatment session, this data is also compared to the latest EEG data to determine the proximity of the positioning cap to the subject's skull. This is accomplished through monitoring of the signals from EEG electrodes and determining which EEG electrodes continue to deliver valid data through-out the treatment session versus those that were delivering valid data at the start of the treatment session and also comparing to those electrodes that delivered valid EEG data during the EEG setup and calibration phase (1200).

It should also be noted that the outputs from the EEG electrodes are selectably processed. That is, electrodes that do not deliver valid data are not processed further. Electrodes that deliver questionable EEG data may be further processed and combined with valid EEG electrode data if this improves the resulting EEG data. Finally, valid EEG data from sensors may be combined and further processes to reduce noise and improve EEG signal integrity.

Similar processing techniques can also be applied to any external EEG electrodes (607) that are analyzed and processed along with the EEG electrode data provided from the EEG electrodes that are part of the positioning cap.

FIG. 13 illustrates one possible version of the EEG calibration and setup screen. In an embodiment, the set up screen includes a schematic representation of the positioning cap (1300); target location (1303); integrated and external EEG electrodes delivering valid signals (1301 and 1305 respectively); EEG electrodes delivering suspect signals (1302); and integrated and external EEG electrodes delivering invalid signals (1304 and 1306 respectively).

A top view is shown, looking through the positioning cap at the EEG electrodes. The target location (505 and 605) is shown as a circle (1303). The array of EEG electrodes on the positioning cap is illustrated such that an empty circle shows an electrode that is not delivering valid data (1304); a solid circle shows an electrode that is delivering valid data (1301); and a “hatched” circle shows an electrode that is delivery questionable data (1302). External electrodes, if used, are illustrated in a similar manner (1305 and 1306). It will be readily recognized that a number of possible variants of this approach are possible and that colors or other indications can be used to indicate EEG electrodes delivering valid, questionable or invalid data.

The positioning system comprised of the location sensor (101); the locator device (105, also FIG. 3 and FIG. 4); the positioning cap (103, also FIGS. 5, 6 and 7); and the positioning device (104, also FIG. 8) operates using techniques that are well known in the art and a number of possible emitter types are possible. Two preferred emitter types are infrared (IR) emitters or ultrasonic emitters. Two high definition video cameras or laser devices may also be used to track the location of the sensors.

IR emitters are readily available as IR light emitting diodes (LEDs) such as the TSAL6400 from Vishay. If IR LEDs are used for emitters, a range of location sensors (101) are available. Two possible location sensors are the VL120:SLIM from Optitrack or the PAC7001CS Object Tracking Sensor (PAC7001CS Object Tracking Sensor—MOT Sensor datasheet—V2.1, May. 2006) from PixArt Imaging. The techniques for position measurement and tracking of IR emitters using triangulation are well known in the art (for example, see Self-calibrating optical object tracking using Wii remotes, Sensors, Cameras, and Systems for Industrial/Scientific Applications X, Proc. SPIE vol. 7249. Calculations for positioning may be implemented in the location sensor (101), on the control unit (108) or on a combination of the location sensor and control unit. If optical targets are used, an imaging camera system can be used, such as the Polaris VICRA camera from Northern Digital Inc. (NDI).

If ultrasonic emitters are used, time of flight measurements and other techniques well-known in the art (e.g., D. Webster, A pulsed ultrasonic distance measurement system based upon phase digitizing, IEEE Transactions on Instrumentation and Measurement, 43 (4), pg. 578-582 will be made on the control unit (108) and the location sensor (101) will consist of off-the-shelf ultrasonic sensor or sensors. Calculations for positioning may be implemented in the location sensor (101), on the control unit (108) or on a combination of the location sensor and control unit.

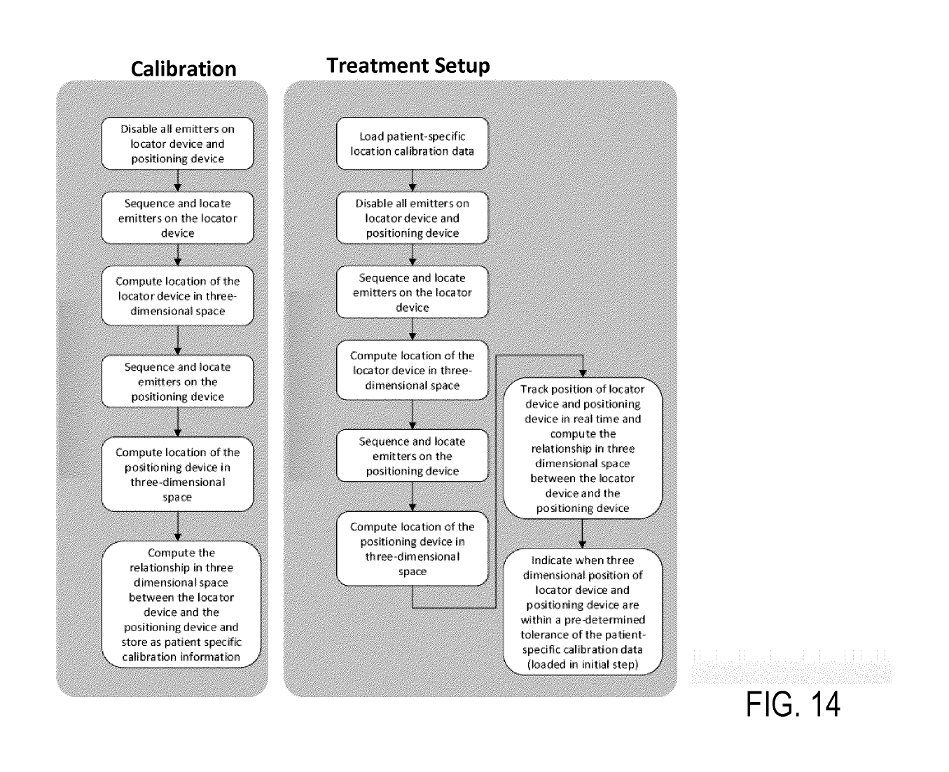

The operation of the positioning system is explained in FIG. 14. In the calibration phase, all emitters (301 or 401) on the locator device (FIG. 3 or FIG. 4) are disabled as are all emitters on the positioning device (803). Then the emitters on the locator device (301 or 401) are independently enabled by the control unit (108) so they can be individually identified and located. Then, using techniques well-known in the art, the position of the locator device in three-dimensional space is computed.

Using a similar approach, the position of the positioning device is computed. Then, the position of the locator device, relative to the positioning device is computed. This information is re-checked with at least one additional position calculation for each of the locator device and the positioning device. Then, the relative position of the locator device and the positioning device is stored as subject specific calibration information in the control unit (108). This information may be stored remotely as part of the subject's electronic medical record or treatment history using the network connection (111). If necessary, the positions of the locator device and the positioning device will be computed relative to each other in real time to ensure that subject movement or other factors do not adversely influence the computation of the relative position measurement between the locator device and the positioning device.

In the treatment setup phase, the subject-specific calibration data that was computed and stored in the calibration phase is loaded by the control unit. As noted above, this information may be retrieved over the network connection (111). Then the emitters on the locator device (301 or 401) are independently enabled by the control unit (108) so they can be individually identified and located. Using techniques well-known in the art, the position of the locator device in three-dimensional space is computed. Using a similar approach, the position of the positioning device is computed. The position of the locator device, relative to the positioning device is computed and compared to the subject-specific position calibration information that was loaded in the first step of the treatment setup phase. If the relative positions of the locator device and positioning device are within a pre-determined tolerance of the subject-specific calibration data, the TMS technician is informed so that the setup phase can complete. If the relative positions of the locator device and positioning device are not within a pre-determined tolerance of the subject-specific calibration data, then the position of the locator device and the positioning device are re-computed at a pre-determined time interval and from this the relative position is again computed and compared to the subject-specific position calibration data loaded in the first step of the treatment setup phase. During the procedure, the TMS technician is guided to locate the positioning cap in the calibrated location using the positioning guidance screen as shown in FIG. 11.

Without loss of generality, the positioning system presented herein which is comprised of the location sensor (101); the locator device (105, also FIG. 3 and FIG. 4); the positioning cap (103, also FIGS. 5, 6 and 7); and the positioning device (104, also FIG. 8) can be extended to support the positioning and integrated EEG monitoring of two or more TMS coils.

Alignment devices (such as locator pins) are designed so that the most focal area of the TMS coil magnetic field aligns with the target location.

In terms of safety features, the system may be configured to monitor brainwave activities of a subject to detect any signs of pre-seizure or seizure. For example, the detected signs may include kindling, a form of pre-epileptic form discharge. The system may be configured to not begin transmission, or to immediately abort transmission upon detection of any signs of pre-seizure or seizure during the application of magnetic stimulation.

Thus, in an aspect, there is provided a method of positioning a treatment device relative to a cranial anatomical location of a subject, comprising: placing a fixed-position locating device on the cranium of the subject; placing an adjustable positioning cap on the cranium of the subject; loading a subject-specific calibration for treatment of a specified cranial anatomical location; determining a three-dimensional position of the fixed-position locator device relative to the adjustable positioning cap; and calibrating the location of the positioning cap by adjusting the positioning cap until it is aligned with the specified cranial anatomical location.

In another aspect, there is provided a system for positioning a treatment device relative to a cranial anatomical location of a subject, the system comprising: a fixed-position locating device for placement on the cranium of the subject; an adjustable positioning cap for placement on the cranium of the subject; processing means for loading a subject-specific calibration for treatment of a specified cranial anatomical location; processing means for determining a three-dimensional position of the fixed-position locator device relative to the adjustable positioning cap; and processing means for guiding the calibration of the location of the positioning cap until it is aligned with the specified cranial anatomical location.

In another aspect, the system is further adapted to monitoring brainwave activities of a subject, and immediately abort transmission upon detection of signs of pre-seizure or seizure during the application of magnetic stimulation. For example, the signs may include kindling, a form of pre-epileptic form discharge. The system may monitor for pre-seizure type activities or kindling prior to applying any magnetic stimulation, and if detected then not begin transmission. The system may also be configured to immediately abort transmission upon detection of a seizure.

Thus, in an aspect, there is provided a method of positioning a treatment device relative to a cranial anatomical location of a subject, comprising: placing a fixed-position locator device on the cranium of the subject; placing an adjustable positioning cap on the cranium of the subject; loading a subject-specific calibration for treatment of a specified cranial anatomical location; determining a three-dimensional position of the fixed-position locator device relative to the adjustable positioning cap; and calibrating the location of the positioning cap by adjusting the positioning cap until it is aligned with the specified cranial anatomical location.

In an embodiment, the locator device includes three or more passive optical targets or three or more active emitters adapted to identify the three-dimensional position of the locator device relative to a location sensor.

In another embodiment, the three or more targets are passive optical targets identifiable by the location sensor.

In another embodiment, three or more emitters are active emitters identifiable by the location sensor.

In another embodiment, the locator device comprises one of glasses or a headband adapted to be worn by the subject so as to repeatedly determine the three-dimensional position of the cranium of the subject relative to the adjustable positioning cap.

In another embodiment, the method further comprises providing a plurality of alignment guides on the adjustable positioning cap, the alignment guides adapted to identify and guide alignment of a target location on the positioning cap.

In another embodiment, the method further comprises providing a plurality of corresponding alignment receptacles for receiving the plurality of alignment guides.

In another embodiment, the method further comprises providing the alignment receptacles in a TMS coil adapted to be mated to the adjustable positioning cap.

In another embodiment, the method further comprises providing an array of electroencephalogram (EEG) electrodes on the adjustable positioning cap.

In another embodiment, the method further comprises monitoring the EEG electrodes to assess the proximity of the TMS coil.

In another aspect, there is provided a system for positioning a treatment device relative to a cranial anatomical location of a subject, the system comprising: a fixed-position locator device for placement on the cranium of the subject; an adjustable positioning cap for placement on the cranium of the subject; processing means for loading a subject-specific calibration for treatment of a specified cranial anatomical location; processing means for determining a three-dimensional position of the fixed-position locator device relative to the adjustable positioning cap; and processing means for guiding the calibration of the location of the positioning cap until it is aligned with the specified cranial anatomical location.

In an embodiment, the locator device includes three or more passive optical targets or three or more active emitters adapted to identify the three-dimensional position of the locator device relative to a location sensor.

In another embodiment, the three or more targets are passive optical targets identifiable by the location sensor.

In another embodiment, the three or more emitters are active emitters identifiable by the location sensor.

In another embodiment, the locator device comprises one of glasses or a headband adapted to be worn by the subject so as to repeatedly determine the three-dimensional position of the cranium of the subject relative to the adjustable positioning cap.

In another embodiment, the system further comprises a plurality of alignment guides on the adjustable positioning cap, the alignment guides adapted to identify and guide alignment of a target location on the positioning cap.

In another embodiment, the system further comprises a plurality of corresponding alignment receptacles for receiving the plurality of alignment guides.

In another embodiment, the system further comprises a TMS coil having alignment receptacles adapted to be mated to the adjustable positioning cap.

In another embodiment, the system further comprises an array of electroencephalogram (EEG) electrodes on the adjustable positioning cap.

In another embodiment, the system monitors the EEG electrodes to assess the proximity of the TMS coil.

While illustrative embodiments of the invention have been described above, it will be appreciate that various changes and modifications may be made without departing from the scope of the present invention.

1. A method of positioning a treatment device relative to a cranial anatomical location of a subject, comprising:
placing a fixed-position locator device on the cranium of the subject;
placing an adjustable positioning cap on the cranium of the subject;
loading a subject-specific calibration for treatment of a specified cranial anatomical location;
determining a three-dimensional position of the fixed-position locator device relative to the adjustable positioning cap; and
calibrating the location of the positioning cap by adjusting the positioning cap until it is aligned with the specified cranial anatomical location.

2. The method of claim 1, wherein the locator device includes three or more passive optical targets or three or more active emitters adapted to identify the three-dimensional position of the locator device relative to a location sensor.

3. The method of claim 2, wherein the three or more targets are passive optical targets identifiable by the location sensor.

4. The method of claim 2, wherein the three or more emitters are active emitters identifiable by the location sensor.

5. The method of claim 2, wherein the locator device comprises one of glasses or a headband adapted to be worn by the subject so as to repeatedly determine the three-dimensional position of the cranium of the subject relative to the adjustable positioning cap.

6. The method of claim 5, further comprising providing a plurality of alignment guides on the adjustable positioning cap, the alignment guides adapted to identify and guide alignment of a target location on the positioning cap.

7. The method of claim 6, further comprising providing a plurality of corresponding alignment receptacles for receiving the plurality of alignment guides.

8. The method of claim 7, further comprising providing the alignment receptacles in a TMS coil adapted to be mated to the adjustable positioning cap.

9. The method of claim 8, further comprising providing an array of electroencephalogram (EEG) electrodes on the adjustable positioning cap.

10. The method of claim 2, further comprising monitoring the EEG electrodes to assess the proximity of the TMS coil.

11. A system for positioning a treatment device relative to a cranial anatomical location of a subject, the system comprising:
a fixed-position locator device for placement on the cranium of the subject;
an adjustable positioning cap for placement on the cranium of the subject;
processing means for loading a subject-specific calibration for treatment of a specified cranial anatomical location;
processing means for determining a three-dimensional position of the fixed-position locator device relative to the adjustable positioning cap; and
processing means for guiding the calibration of the location of the positioning cap until it is aligned with the specified cranial anatomical location.

12. The system of claim 11, wherein the locator device includes three or more passive optical targets or three or more active emitters adapted to identify the three-dimensional position of the locator device relative to a location sensor.

13. The system of claim 12, wherein the three or more targets are passive optical targets identifiable by the location sensor.

14. The system of claim 12, wherein the three or more emitters are active emitters identifiable by the location sensor.

15. The system of claim 12, wherein the locator device comprises one of glasses or a headband adapted to be worn by the subject so as to repeatedly determine the three-dimensional position of the cranium of the subject relative to the adjustable positioning cap.

16. The system of claim 15, further comprising a plurality of alignment guides on the adjustable positioning cap, the alignment guides adapted to identify and guide alignment of a target location on the positioning cap.

17. The system of claim 16, further comprising a plurality of corresponding alignment receptacles for receiving the plurality of alignment guides.

18. The system of claim 17, further comprising a TMS coil having alignment receptacles adapted to be mated to the adjustable positioning cap.

19. The system of claim 18, further comprising an array of electroencephalogram (EEG) electrodes on the adjustable positioning cap.

20. The system of claim 19, wherein the EEG electrodes are monitored to assess the proximity of the TMS coil.
